(19) United States
(12) Patent Application Publication
(10) Pub. No.: US 2013/0108701 A1
(43) Pub. Date: May 2, 2013

(54) Solid Dosage Forms of Antipsychotics

(71) Applicants: Krishna Murthy Bhavanasi, Hyderabad (IN); Nagaprasad Vishnubhotla, Hyderabad (IN); Sivakumaran Meenakshisunderam, Hyderabad (IN)

(72) Inventors: Krishna Murthy Bhavanasi, Hyderabad (IN); Nagaprasad Vishnubhotla, Hyderabad (IN); Sivakumaran Meenakshisunderam, Hyderabad (IN)

(21) Appl. No.: 13/261,522

(22) PCT Filed: May 23, 2011
(86) PCT No.: PCT/IB2011/001123

§ 371 Date: Nov. 20, 2012

(30) Foreign Application Priority Data
May 25, 2010 (IB) 1442/CHE/2010

(51) Int. Cl. A61K 9/16

(52) U.S. Cl. CPC A61K 9/1676; A61K 9/1605; A61K 9/1682
USPC 424/490; 514/254.04; 264/128

(57) ABSTRACT

The present invention relates to a solid dosage form comprising an antipsychotic, particularly ziprasidone. The present invention also relates to a process for preparation of solid dosage form comprising ziprasidone.

### FIELD OF THE INVENTION

The present invention relates to a solid dosage form comprising an antipsychotic, particularly ziprasidone. The present invention also relates to a process for preparation of solid dosage form comprising ziprasidone.

### BACKGROUND OF THE INVENTION

Ziprasidone is an atypical antipsychotic, chemically known as 5-[2-[4-(1,2-benzisothiazol-3-yl)-1-piperazinyl]ethyl]-6-chloro-1,3-dihydro-2H-indol-2-one and is first disclosed in U.S. Pat. No. 4,831,031. Ziprasidone is currently marketed in the United States under the trade name GEODON®, in the form of oral capsules, oral suspension and intramuscular injection. The oral capsules and oral suspension contain ziprasidone hydrochloride monohydrate which is disclosed in U.S. Pat. No. 5,312,925. Ziprasidone hydrochloride has high permeability but possess relatively poor aqueous solubility, a factor which unfavorably affects bioavailability.

Low-solubility drugs often show poor bioavailability or irregular absorption, the degree of irregularity being affected by factors such as dose level, fed state of the patient, and polymorphic nature of the drug. Increasing the bioavailability of low-solubility drugs has been the subject of much research. Typical approaches can involve: (1) using particular formulation excipients, which increase solubility, for example surfactants; and/or (2) formulating the drug in a small particle size, thereby increasing the surface area of the drug to facilitate more rapid dissolution. Manipulating the particle size can present technical difficulties and quality control challenges.

U.S. Pat. No. 4,831,031 discloses the preparation of ziprasidone and salts thereof. As per the disclosure in U.S. 2008/0268034, ziprasidone hydrochloride is obtained in a very fine particle size by following the process disclosed in U.S. '031 patent. It would be advantageous to use ziprasidone in fine particle size form obtained as per process disclosed in U.S. '031 patent for formulating dosage forms as this procedure would reduce at least one process step. The '034 patent publication further discloses that ziprasidone of small particle size is fluffy and tends to agglomerate due to surface charge, which decreases the effective available surface area.

The decrease in effective surface area results in slowed dissolution of ziprasidone contrary to the expectation that decreased particle size would enhance the solubility. Ziprasidone tends to form agglomerates when it comes in contact with an aqueous liquid. The agglomerates may reduce the dissolution of ziprasidone when the dosage form is in contact with gastrointestinal fluids. The agglomerates further contribute to handling problems while formulating a dosage form. This also leads to problems of content uniformity in the dosage forms and reproducibility of dissolution profile.

There are several prior art references which discloses various attempts to improve the solubility of ziprasidone, to prevent the agglomeration of ziprasidone while formulating different compositions comprising ziprasidone.

U.S. Pat. No. 6,150,366 discloses a composition comprising crystalline ziprasidone free base or ziprasidone hydrochloride having a mean particle size equal to or less than about 85 μm which exhibit good dissolution properties at physiologic pH. The patent further discloses that ziprasidone having a particle size of at least at or below 85 μm has a dissolution rate in aqueous media that does not vary substantially with the particle size, and therefore appears to be largely independent of particle size in this range.

U.S. Pat. No. 6,232,304 and U.S. Pat. No. 6,399,777 discloses inclusion complexes of ziprasidone with cyclodextrin to improve the solubility.

U.S. Pat. No. 6,548,555 and U.S. 2005/0049223 discloses a composition comprising a basic drug, a drug which forms a zwitterion,, or a salt of either, admixed with a polymer selected from hydroxypropyl methylcellulose acetate succinate (HPMCAS), cellulose acetate trimellitate (CAT), cellulose acetate phthalate (CAP), hydroxypropyl cellulose acetate phthalate (HPCAP), hydroxypropyl methylcellulose acetate phthalate (HPMCAP), and methylcellulose acetate phthalate (MCAP) to increase the dissolution of the drug.

U.S. 2002/0006443, U.S. 2003/0072801, U.S. 2003/0224043 and U.S. 2003/0228358 discloses a composition comprising (1) a drug in a solubility-improved form selected from the group consisting of drug in nanoparticulate form, absorbed drug, drug in a nanosuspension, a supercooled melt of drug, cyclodextrin/drug form, gelatin form, softgel form, self-emulsifying form, and three-phase drug form and (2) a concentration-enhancing polymer selected from the group consisting of hydroxypropyl methyl cellulose acetate succinate, hydroxypropyl methyl cellulose phthalate, cellulose acetate phthalate, cellulose acetate trimellitate, carboxymethyl ethyl cellulose, hydroxypropyl methyl cellulose, poloxamers, polyvinylpyrrolidone, polyvinyl alcohols that have at least a portion of their repeat units in hydrolyzed-form, and mixtures thereof, which provides 1.25-fold concentration of the drug in the use environment to the concentration of drug when the polymer is not present.

U.S. 2002/0009494 and U.S. 2008/0262107 discloses composition comprising a spray dried solid dispersion comprising a sparingly water-soluble drug and hydroxypropyl methylcellulose acetate succinate (HPMCAS), said dispersion providing a maximum concentration of said drug in a use environment that is higher by a factor of at least 1.5 relative to a control composition comprising an equivalent quantity of undispersed drug.

U.S. 2003/0054037 discloses a pharmaceutical composition comprising a solid adsorbate with a low-solubility drug adsorbed onto a substrate, said substrate having a surface area of at least 20 m²/g, wherein at least a major portion of said drug in said adsorbate is amorphous.

U.S. 2003/0104063 discloses a pharmaceutical composition comprising: (a) a solid dispersion comprising a low-solubility drug and a matrix, wherein at least a major portion of said drug in said dispersion is amorphous; and (b) a concentration-enhancing polymer, said dispersion being free from at least a portion of said concentration-enhancing polymer.

U.S. 2003/0170309 discloses a pharmaceutical composition comprising solid aggregated polymer/drug assemblies, said solid aggregated polymer/drug assemblies comprising a low-solubility drug and an amphiphilic polymer and said drug being present in said solid aggregated polymer/drug assemblies in a semi-ordered, non-crystalline state.

U.S. 2004/0142039 discloses a solid ionic conjugate comprising a pharmaceutical compound and a functional polymer, said solid ionic conjugate having aqueous solubility greater than that of said pharmaceutical compound.

U.S. 2004/0147532 discloses a liquid conjugate comprising a bioactive agent and an absorbable liquid polymer, said bioactive agent and said absorbable liquid polymer being at least partly ionically linked together to form said liquid conjugate.

U.S. 2005/0049295 discloses a pharmaceutical composition comprising ziprasidone hydrochloride having a mean particle size of greater than about 85 micron and less than about 300 micron and a pharmaceutically acceptable carrier.

U.S. 2005/0163858 discloses a formulation comprising ziprasidone or a pharmaceutically acceptable salt thereof, wherein the active agent has a mean particle size greater than 85 micrometers and a pharmaceutically acceptable carrier and further discloses controlled release dosage form comprising ziprasidone, This patent publication further discloses that the formulation comprising ziprasidone having a mean particle size greater than 85 micrometers is bioequivalent to GEODON®.

U.S. 2005/0249814 discloses a pharmaceutical composition having improved solubility comprising a hydrophobic drug or pharmaceutically acceptable salt thereof and a compound having at least one carboxylic acid moiety, wherein the molar ratio of the compound having at least one carboxylic acid moiety to the hydrophobic drug or pharmaceutically acceptable salt thereof is from about 0.1:1 to about 25:1.

U.S. 2007/0237828 discloses a dosage form comprising ziprasidone or a salt thereof in the form of particles having a mean size at least about 90 μm and a hydrophilic excipient, and having a ziprasidone bioavailability equal to or greater than the bioavailability of a dosage form where ziprasidone or a salt thereof is present as particles having a mean size less than 85 μm. This patent publication further discloses that by incorporating hydrophilic excipients in the pharmaceutical compositions, the aqueous solubility of ziprasidone and its salts is increased. The hydrophilic excipients are thought to act by decreasing surface tension and thereby forming micelles that assist in the solubilization of ziprasidone or the salt having particle sizes greater than about 90 μm.

U.S. 2008/0268034 discloses a dosage form comprising ziprasidone having a particle size D90 less than or equal to 10 μm, colloidal silicon dioxide in a weight ratio with the ziprasidone of about 1:0.1 to 1:1, and optionally one or more pharmaceutically acceptable excipients. This patent publication further discloses that ziprasidone having less particle size tends to form agglomerates, and colloidal silicon dioxide, when mixed with these agglomerate, tends to neutralize the surface charges from the particles and prevents agglomeration.

U.S. 2008/0286373 discloses a ziprasidone formulation containing at least (a) one ziprasidone compound and at least an excipient component (b) that includes at least one of (i) one or more of a mono-, di-, or tri-ester of C₁₂₋₂₄ fatty acids and glycerol, in which each fatty acid group is chosen independently of the others, or mixtures thereof; and/or (ii) one or more mono- or di-esters of C₁₂₋₂₄ fatty acids and polyC₂₋₃alkyleglycol, in which each fatty acid group is chosen independently of the others, or mixtures thereof; and/or (iii) a vitamin E TPGS (Vitamin E tocopherol-succinic acid-polyethylene glycol); the formulation further comprising (c) at least one surfactant selected from anionic and non-nonionic surfactants and still further comprising (d) at least one hydroxyalkyl alkylcellulose in which each alkyl group and each hydroxyalkyl group independently has from 1 to 4 carbon atoms.

U.S. 2009/0142404 discloses a dosage form comprising a low-solubility drug and a precipitation-inhibiting polymer. The low-solubility drug is in a solubility-improved form and in the form of particles at least partially coated with the precipitation-inhibiting polymer.

WO 2004/0054621 discloses water-soluble clathrates of ziprasidone comprising ziprasidone and its salts as active principle, and derivatives of cyclodextrins as clathration materials.

WO 2007/027273 discloses a nanoparticulate composition comprising ziprasidone and at least one surface stabilizer.

WO 2007/102038 discloses an oral pharmaceutical composition comprising therapeutically effective amount of ziprasidone hydrochloride hydrate or its salt thereof having a mean particle size in the range of 100-300 μm along with other suitable pharmaceutical excipients to enhance stability and dissolution properties at physiologic pH.

WO 2007/126322 discloses a method for obtaining a pharmaceutical composition comprising ziprasidone or a pharmaceutically acceptable salt thereof, to improve solubility and thus bioavailability of the drug, whereby the active agent is slugged together with one or more pharmaceutically acceptable excipients.

WO 2010/025848 discloses a pharmaceutical composition for oral administration, which comprises particles, which are produced by compacting and granulating a mixture containing ziprasidone or a pharmaceutically acceptable salt thereof as an active ingredient and a disintegrant, so that an intimate contact of the active ingredient particles with the disintegrant is given.

WO 2010/082855 discloses a pharmaceutical composition containing ziprasidone as an active ingredient and pharmaceutically acceptable carrier, wherein the active ingredient is ziprasidone in micronized form and pharmaceutically acceptable carrier is a substance of high capillary activity able to form gel in aqueous environment.

WO 2011/018801 discloses a solid oral dosage form comprising ziprasidone or its pharmaceutically acceptable salt, cationic ion exchange resin and optionally pharmaceutical excipient(s).

IN 3099/CHE/2008 discloses a ziprasidone ion-exchange complex comprising ziprasidone or its pharmaceutically acceptable salt thereof and an ion exchange resin.

IN 1061/CHE/2010 discloses a pharmaceutical composition comprising ziprasidone or a pharmaceutically acceptable salt thereof, starch phosphate and optionally one or more pharmaceutically acceptable excipients.

The above prior art references disclose various attempts to improve the solubility of ziprasidone and to prevent the agglomeration of ziprasidone particles for proper handling during formulation. There exists an unmet need to develop a solid dosage form comprising ziprasidone having better dissolution properties, better content uniformity and better or equivalent bioavailability w.r.t commercialized ziprasidone dosage form. The inventors of the present invention have developed a solid dosage form comprising ziprasidone which provides desired dissolution properties, content uniformity and bioavailability.

The solid dosage form according to the present invention comprises ziprasidone in both intragranular component and extragranular component which provides better content uniformity irrespective of particle size. Further the process for preparation of solid dosage form according to present invention provides better distribution of drug throughout the solid dosage form, desired dissolution of ziprasidone and prevents agglomeration of ziprasidone.

### OBJECTIVE OF THE INVENTION

The main objective of the present invention is to provide a solid dosage form comprising ziprasidone and one or more pharmaceutically acceptable excipients.

Another objective of the present invention is to provide a process for the preparation of solid dosage form comprising ziprasidone having better dissolution properties, content uniformity and equivalent bioavailability w.r.t commercialized ziprasidone dosage form.

### SUMMARY OF THE INVENTION

The present invention relates to a solid dosage form comprising:
a) intragranular component comprising ziprasidone, binder and optionally one or more pharmaceutically acceptable excipients; and
b) extragranular component comprising ziprasidone and one or more pharmaceutically acceptable excipients.

The present invention further relates to a solid dosage form comprising:
a) intragranular component comprising an inert core, a coating layer comprising ziprasidone and a binder, and optionally one or more pharmaceutically acceptable excipients, and
b) extragranular component comprising ziprasidone and one or more pharmaceutically acceptable excipients.

Accordingly, the present invention provides a process for the preparation of solid dosage form comprising ziprasidone, which comprises the steps of:
a) dispersing ziprasidone in a binder solution,
b) granulating one or more pharmaceutically acceptable excipients with dispersion of step a),
c) blending the granules with one or more extragranular excipients and optionally ziprasidone, and
d) processing into a solid dosage form.

### DETAILED DESCRIPTION OF THE INVENTION

The present invention relates to a solid dosage form comprising ziprasidone and one or more pharmaceutically acceptable excipients.

The present invention further relates to a solid dosage form comprising:
a) intragranular component comprising ziprasidone, binder and optionally one or more pharmaceutically acceptable excipients; and
b) extragranular component comprising ziprasidone and one or more pharmaceutically acceptable excipients.

The present invention further relates to a solid dosage form comprising:
a) intragranular component comprising ziprasidone, binder and optionally one or more pharmaceutically acceptable excipients; and
b) extragranular component comprising ziprasidone and one or more pharmaceutically acceptable excipients,
wherein the ratio of intragranular ziprasidone to extragranular ziprasidone is the range of 1:1 to 1:10.

The present invention further relates to a solid dosage form comprising:
a) intragranular component comprising an inert core, a coating layer comprising ziprasidone and a binder, and optionally one or more pharmaceutically acceptable excipients, and
b) extragranular component comprising ziprasidone and one or more pharmaceutically acceptable excipients.

The present invention further relates to a solid dosage form comprising:
a) intragranular component comprising an inert core, a coating layer comprising ziprasidone and a binder, and optionally one or more pharmaceutically acceptable excipients, and
b) extragranular component comprising ziprasidone and one or more pharmaceutically acceptable excipients,
wherein the ratio of intragranular ziprasidone to extragranular ziprasidone is the range of 1:1 to 1:10.

The present invention further relates to a process for the preparation of solid dosage form comprising ziprasidone, which comprises the steps of:
a) dispersing intragranular ziprasidone in a binder solution,
b) granulating one or more pharmaceutically acceptable excipients with dispersion of step a),
c) blending the granules with extragranular ziprasidone and one or more extragranular excipients, and
d) processing into a solid dosage form.

The present invention further relates to a process for the preparation of solid dosage form comprising ziprasidone, which comprises the steps of:
a) dispersing intragranular ziprasidone in a binder solution,
b) granulating one or more pharmaceutically acceptable excipients with dispersion of step a),
c) blending the granules with extragranular ziprasidone and one or more extragranular excipients, and
d) processing into a solid dosage form
wherein the ratio of intragranular ziprasidone to extragranular ziprasidone is in the range of 1:1 to 1:10.

The present invention further relates to a solid dosage form comprising ziprasidone and one or more pharmaceutically acceptable excipients wherein the solid dosage form is prepared by the process comprising the steps of:
a) dispersing intragranular ziprasidone in a binder solution,
b) granulating one or more pharmaceutically acceptable excipients with dispersion of step a),
c) blending the granules with extragranular ziprasidone and one or more extragranular excipients, and
d) processing into a solid dosage form.

The present invention further relates to a solid dosage form comprising ziprasidone prepared by a process comprising the steps of:
a) preparing binder solution by dissolving the binder in a suitable solvent,
b) dispersing intragranular ziprasidone in the binder solution,
c) granulating a diluent with the drug suspension of step (b),
d) blending the granules of step (c) with extragranular ziprasidone and one or more extragranular excipients, and
e) lubricating the blend of step (d) and processing into a solid dosage form.

Ziprasidone according to the present invention includes, but not limited to, Ziprasidone free base, its pharmaceutical acceptable salts, esters, ethers, solvates, hydrates, polymorphs and the like. Ziprasidone may be used in the range of 1-50% by weight of the composition.

Examples of pharmaceutically acceptable salts include, but are not limited to, mineral or organic acid addition salts of basic residues such as amines; alkali or organic addition salts of acidic residues such as carboxylic acids; and the like, and combinations comprising one or more of the foregoing salts. The pharmaceutically acceptable salts include non-toxic salts and the quaternary ammonium salts of the parent compound formed, for example, from non-toxic inorganic or organic acids. For example, non-toxic acid salts include those derived from inorganic acids such as hydrochloric, hydrobromic, sulfuric, sulfamic, phosphoric, nitric and the like; other acceptable inorganic salts include metal salts such as sodium salt, potassium salt, cesium salt, and the like; and alkaline earth metal salts, such as calcium salt, magnesium salt, and the like, and combinations comprising, one or more of the foregoing salts. Pharmaceutically acceptable organic salts includes salts prepared from organic acids such as acetic, pamoic, maleic, hydroxymaleic, phenylacetic, glutamic, benzoic, salicylic, mesylic, esylic, besylic, sulfanilic, 2-acetoxybenzoic, fumaric, toluenesulfonic, oxalic, and the like; organic amine salts such as triethylamine salt, pyridine salt, picoline salt, ethanolamine salt, triethanolamine salt, dicyclohexylamine salt, N,N′-dibenzylethylenediamine salt, and the like; and amino acid salts such as arginate, asparginate, glutamate, and the like; and combinations comprising one or more of the foregoing salts. Preferably the pharmaceutically acceptable salts include hydrochloride, mesylate, tosylate, esylate, tartrate, napsylate, besylate, aspartate and the like.

“Pharmaceutically acceptable excipient/s” are components added to active agent pharmaceutical formulation other than the ziprasidone. Excipients may be added to facilitate manufacture, enhance stability, control release, enhance product characteristics, enhance bioavailability, enhance patient acceptability, etc. Pharmaceutically acceptable excipients includes, but not limited to, diluents/fillers, binders, disintegrants, lubricants, glidants, compression aids, colors, sweeteners, preservatives, surfactants, suspending agents, dispersing agents, film formers, flavors, printing inks, etc.

Binders hold the ingredients in the composition together. Exemplary binders include, but not limited to, cellulose and its derivatives including, ethyl cellulose, hydroxypropyl cellulose, hydroxypropyl methylcellulose, methylcellulose and hydroxyethyl cellulose, carboxymethyl cellulose; starch and its derivatives; hydrocolloids; sugars; polyvinyl pyrrolidone and combinations comprising one or more of the foregoing binders. The binder may be used in the range of 1-15% by weight of the composition.

Diluents increase the bulk of the composition. Diluents according to the present invention include, but not limited to, sugars such as lactose, sucrose; dextrose; sugar alcohols such as mannitol, sorbitol, xylitol, lactitol; Starlac, Microcelac, starch, modified starches, dibasic calcium phosphate, tribasic calcium phosphate, powdered cellulose, microcrystalline cellulose, silicified microcrystalline cellulose and the like or combinations thereof. The diluent may be used in the range of 5-80% by weight of the composition.

Disintegrants according to the present invention include, but not limited to, water swellable substances, for example, cellulose and its derivatives including low-substituted hydroxypropyl cellulose; cross-linked polyvinylpyrrolidone; cross-linked sodium carboxymethylcellulose, sodium carboxymethylcellulose, microcrystalline cellulose; sodium starch glycolate; ion-exchange resins; starch and modified starches including pregelatinized starch; formalin-casein; and combinations comprising one or more of the foregoing water swellable substances. The disintegrant may be used in the range of 1-20% by weight of the composition.

Lubricants and glidants aids in the processing of powder materials. Exemplary lubricants include, but not limited to, calcium stearate, glycerol behenate, magnesium stearate, mineral oil, polyethylene glycol, sodium stearyl fumarate, stearic acid, talc, vegetable oil, zinc stearate, and combinations comprising one or more of the foregoing lubricants. Exemplary glidants include, but not limited to, talc, silicon dioxide, cornstarch and the like. The lubricant may be used in the range of 0.1-5% by weight of the composition.

Surfactants are compounds which are capable of improving the wetting of the drug and/or enhancing the dissolution. The surfactants can be selected from hydrophilic surfactants or lipophilic surfactants or mixtures thereof. The surfactants can be anionic, nonionic, cationic, and zwitterionic surfactants. Surfactants according to the present invention include, but not limited to, polyoxyethylene alkylaryl ethers such as polyoxyethylene lauryl ether, polyoxyethylene cetyl ether, polyoxyethylene stearyl ether; polyethylene glycol fatty acid esters such as PEG monolaurate, PEG dilaurate, PEG distearate, PEG dioleate; polyoxyethylene sorbitan fatty acid ester such as polysorbate 40, polysorbate 60, polysorbate 80; sorbitan fatty acid mono esters such as sorbitan monolaurate, sorbitan monooleate, sorbitan sesquioleate, sorbitan trioleate, polyoxyethylene castor oil derivates such as polyoxyl castor oil, polyoxyl hydrogenated castor oil, sodium lauryl sulphate and the like or combinations thereof. The surfactant may be used in the range of 5-20% by weight of the composition.

“Suitable solvent” according to the present invention can be any solvent in which the binder is soluble or dispersible and is selected from isopropyl alcohol, ethanol, water, acetone, methylene chloride and the like or mixtures thereof.

In a preferred embodiment of the present invention, the solid dosage form comprises 1-50% by weight of ziprasidone, 1-15% by weight of binder selected from ethyl cellulose, hydroxypropyl methylcellulose and polyvinyl pyrrolidone, 5-80% by weight of diluent selected from lactose, mannitol and microcrystalline cellulose, 1-20% by weight of disintegrant selected from pregelatinized starch, sodium starch glycolate and crospovidone, 0.1-5% by weight of lubricant selected from talc and magnesium stearate.

In another preferred embodiment of the present invention, the solid dosage form comprises:
a) intragranular component comprising 1-40% by weight of ziprasidone, 1-15% by weight of ethyl cellulose, 1-20% by weight of lactose,
b) an extragranular component comprising 5-40% by weight of ziprasidone, 5-60% by weight of lactose, 1-20% by weight of pregelatinized starch, 0.1-5% by weight of magnesium stearate.

In another preferred embodiment, the solid dosage form comprising ziprasidone is prepared by a process comprising the steps of:
a) preparing binder solution by dissolving ethyl cellulose in a mixture of water and isopropyl alcohol,
b) dispersing intragranular ziprasidone in the binder solution,
c) granulating intragranular lactose with the ziprasidone suspension of step (b),
d) blending the granules of step (c) with extragranular ziprasidone, extragranular lactose, pregelatinized starch,
e) lubricating the blend of step (d) with magnesium stearate, and
f) filling the lubricated blend into a capsule shell.

In another embodiment, the particle size of ziprasidone or its pharmaceutically acceptable salts used is in the range of about 5 microns to about 200 microns.

In another embodiment, the ratio of intragranular ziprasidone to extragranular ziprasidone is in the range of 1:1 to 1:10, more preferably in the range of 1:1 to 1:5.

In yet another embodiment, the amount of ziprasidone used may be in the range from about 5 to about 200 mg.

In another embodiment of the present invention, the solid dosage form comprising ziprasidone may be in the form of tablet, capsule, powder, dispersible granules, pellets, beads or the like.

The following examples further exemplify the invention and are not intended to limit the scope of the invention. It is obvious to those skilled in the art to find out the composition for other dosage forms and substitute the equivalent excipients as described in this specification or with the one known to the industry.

### EXAMPLE 1

| S. No | Ingredients | mg/cap |
|---|---|---|
| | Intragranular | |
| 1 | Ziprasidone hydrochloride | 6.53 |
| 2 | Ethyl cellulose | 6.00 |
| 3 | Lactose monohydrate | 9.50 |
| 4 | Isopropyl alcohol | Q.S |
| 5 | Purified water | Q.S |
| | Extragranular | |
| 6 | Ziprasidone hydrochloride | 15.85 |
| 7 | Lactose monohydrate | 46.62 |
| 8 | Pregelatinized starch | 7.00 |
| 9 | Magnesium stearate | 1.00 |
| | Total weight | 92.50 |

The processing steps involved in manufacturing solid dosage form comprising ziprasidone were given below:
i) ziprasidone hydrochloride and lactose monohydrate were sifted,
ii) ethyl cellulose was dissolved in isopropyl alcohol and stirred to get a clear solution,
iii) purified water was added to the solution of step (ii),
iv) ziprasidone hydrochloride was dispersed into the solution of step (iii) and stirred to get a uniform dispersion,
v) sifted lactose was granulated using the drug dispersion of step (iv) and the granules were dried,
vi) the granules obtained in step (v) were blended with the extragranular ziprasidone hydrochloride, lactose monohydrate and pregelatinized starch,
vii) the blend of step (vi) was lubricated with magnesium stearate, and
viii) the lubricated blend of step (vii) was filled into capsule shell.

### EXAMPLE 2

| S. No | Ingredients | mg/cap |
|---|---|---|
| | Intragranular | |
| 1 | Ziprasidone hydrochloride | 6.53 |
| 2 | Ethyl cellulose | 4.50 |
| 3 | Lactose monohydrate | 11.00 |
| 4 | Isopropyl alcohol | Q.S |
| 5 | Purified water | Q.S |
| | Extragranular | |
| 6 | Ziprasidone hydrochloride | 15.85 |
| 7 | Lactose monohydrate | 44.12 |
| 8 | Pregelatinized starch | 7.00 |
| 9 | Magnesium stearate | 1.00 |
| | Total weight | 90.00 |

### EXAMPLE 3

| S. No | Ingredients | mg/cap |
|---|---|---|
| | Intragranular | |
| 1 | Ziprasidone hydrochloride | 6.53 |
| 2 | Ethyl cellulose | 3.75 |
| 3 | Lactose monohydrate | 11.75 |
| 4 | Isopropyl alcohol | Q.S |
| 5 | Purified water | Q.S |
| | Extragranular | |
| 6 | Ziprasidone hydrochloride | 15.85 |
| 7 | Lactose monohydrate | 44.12 |
| 8 | Pregelatinized starch | 7.00 |
| 9 | Magnesium stearate | 1.00 |
| | Total weight | 90.00 |

### EXAMPLE 4

| S. No | Ingredients | mg/cap |
|---|---|---|
| | Intragranular | |
| 1 | Ziprasidone hydrochloride | 6.53 |
| 2 | Ethyl cellulose | 3.00 |
| 3 | Lactose monohydrate | 12.50 |
| 4 | Isopropyl alcohol | Q.S |
| 5 | Purified water | Q.S |
| | Extragranular | |
| 6 | Ziprasidone hydrochloride | 15.85 |
| 7 | Lactose monohydrate | 44.12 |
| 8 | Pregelatinized starch | 7.00 |
| 9 | Magnesium stearate | 1.00 |
| | Total weight | 90.00 |

The compositions given in Examples 2 to 4 were prepared using the similar procedure described in Example 1.

Table 1 given below shows the comparative dissolution profile of ziprasidone capsules of the present invention and Geodon® capsules carried out in 900 ml of pH 7.5 phosphate buffer+2% SLS using Apparatus USP II (Paddle), @ 75 rpm speed. The release profile (% of drug released) was given in Table 1.

TABLE 1

| | % Drug released | | | | |
|---|---|---|---|---|---|
| Time in min | Example-1 | Example-2 | Example-3 | Example-4 | Geodon ® |
| 10 | 52 | 52 | 55 | 59 | 59 |
| 15 | 58 | 61 | 66 | 71 | 73 |
| 20 | 64 | 68 | 72 | 75 | 86 |
| 30 | 71 | 76 | 80 | 82 | 93 |
| 45 | 76 | 83 | 87 | 88 | 95 |
| 60 | 81 | 88 | 90 | 92 | 96 |

We claim:

1. A solid dosage form comprising:
a) intragranular component comprising ziprasidone, binder and optionally one or more pharmaceutically acceptable excipients; and
b) extragranular component comprising ziprasidone and one or more pharmaceutically acceptable excipients.

2. The solid dosage form according to claim 1 wherein
intragranular component comprises an inert core, a coating layer comprising ziprasidone and a binder, and optionally one or more pharmaceutically acceptable excipients.

3. The solid dosage form according to claim 1, wherein one or more pharmaceutically acceptable excipients are selected from the group comprising disintegrant, diluent, lubricant, glidant, and binder.

4. The solid dosage form according to claim 1, wherein the ratio of intragranular ziprasidone to extragranular ziprasidone is in the range of 1:1 to 1:10.

5. The solid dosage form according to claim 3, wherein the diluent is selected from sugars, sugar alcohols, starch, modified starches, dibasic calcium phosphate, tribasic calcium phosphate, powdered cellulose, microcrystalline cellulose and silicified microcrystalline cellulose and combination thereof.

6. (canceled)

7. The solid dosage form according to claim 3, wherein the binder is selected from cellulose and its derivatives, starch and its derivatives, hydrocolloids, sugars, polyvinyl pyrrolidone and combination thereof.

8. (canceled)

9. The solid dosage form according to claim 3, wherein the disintegrant is selected from cellulose and its derivatives, low-substituted hydroxypropyl cellulose; cross-linked polyvinylpyrrolidone; cross-linked sodium carboxymethylcellulose, sodium carboxy methylcellulose, microcrystalline cellulose; sodium starch glycolate; ion-exchange resins;
starch and modified starches, formalin-casein and combination thereof.

10. The solid dosage form according to claim 3, wherein the lubricant is selected from calcium stearate, glycerol behenate, magnesium stearate, mineral oil, polyethylene glycol, sodium stearyl fumarate, stearic acid, talc, vegetable oil, zinc stearate and combination thereof.

11. A process for the preparation of solid dosage form comprising ziprasidone, which comprises the steps of:
a) dispersing ziprasidone in a binder solution,
b) granulating one or more pharmaceutically acceptable excipients with dispersion of step a),
c) blending the granules with extragranular ziprasidone and one or more extragranular excipients, and
d) processing into a solid dosage form.

12. The solid dosage form comprising:
a) intragranular component comprising 1-40% by weight of ziprasidone, 1-15% by weight of ethyl cellulose, 1-20% by weight of lactose,
b) extragranular component comprising 5-40% by weight of ziprasidone, 5-60% by weight of lactose, 1-20% by weight of pregelatinized starch, 0.1-5% by weight of magnesium stearate.
